SEQUENCE LISTING (2 sequences)

SEQ ID NO: 1 - DNA, 198 bp, Artificial Sequence
  Description of Artificial Sequence Synthetic construct
atgagctccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaagaacaaaaactcatctcagaagaggatctg

SEQ ID NO: 2 - DNA, 4200 bp, Artificial Sequence
  Description of Artificial Sequence Synthetic construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccatgagctccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaagaacaaaaactcatctcagaagaggatctgtaggcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

(19) United States
(12) Patent Application Publication
(10) Pub. No.: US 2007/0015232 A1
(43) Pub. Date: Jan. 18, 2007

(54) Mitotic index assay

(75) Inventors: Keith R. Olson, Pleasanton, CA (US); Peter A. Fung, Sunnyvale, CA (US); Richard M. Eglen, Los Altos, CA (US)

Correspondence Address: PETERS VERNY JONES & SCHMITT, L.L.P., 425 SHERMAN AVENUE, SUITE 230, PALO ALTO, CA, 94306, US

(73) Assignee: DiscoveRx, Inc., Fremont, CA (US)

(21) Appl. No.: 11/471,694

(22) Filed: Jun. 20, 2006

Related U.S. Application Data
(60) Provisional application No. 60/692,927, filed on Jun. 21, 2005.

(51) Int. Cl. C12Q 1/34

(52) U.S. Cl. 435/18

(57) ABSTRACT

Mitosis of cells is determined, particularly in the presence of a candidate agent, using cells comprising members of an enzyme fragmentation complex pair, where one of the members is in the nucleus and the other member is in the cytoplasm. By growing the cells where mitosis may occur, one adds a substrate providing a detectable product, where the production of the detectable product is indicative of mitosis.

### CROSS-REFERENCE TO RELATED APPLICATIONS

This application claims priority from U.S. Provisional Patent Application No. 60/692,927, filed on Jun. 21, 2005, entitled “Mitotic Index Assay,” which is hereby incorporated by reference in its entirety.

### STATEMENT OF GOVERNMENTAL SUPPORT

None

### REFERENCE TO SEQUENCE LISTING, COMPUTER PROGRAM, OR COMPACT DISK

Applicants assert that the paper copy of the Sequence Listing is identical to the Sequence Listing in computer readable form found on the accompanying computer disk. Applicants incorporate the contents of the sequence listing by reference in its entirety.

### BACKGROUND OF THE INVENTION

1. Field of the Invention

The present invention relates to the field of assays carried out in cells, and particularly to assay for monitoring mitotic index in cultured cell lines.

2. Related Art

Cell cultures find application in a wide variety of ways. In many studies of cellular pathways, responses to external stimuli, cell proliferation, and the like, the cell population is in different stages of the mitotic cycle. Therefore, the cellular composition of the cells at the different stages of the mitotic cycle will be different. Also, the number of cells will be varying as to proliferation and cell death. In these studies there is an interest in knowing over a period of time, how many cells underwent mitosis as compared to dying or being dormant.

One area of interest is to know whether cells actively proliferating respond differently from cells that are dormant. Depending upon the nature of the cells, the cells may be of a kind that actively regenerates in vivo, such as blood cell progenitors, epithelial cells, endothelial cells, etc. Other types of cells do not actively regenerate in vivo, such as brain cells, pancreatic cells, cardiomyocytes, etc. Whether these cells under the culture conditions proliferate or remain dormant is important in understanding the effects of external stimuli on the mitotic cycle.

In determining the effect of drugs on cells in culture, there will frequently be interest in knowing the degree of proliferation of the cells during the test. One can simultaneously compare a culture comprising a drug and a comparable culture in which the drug is absent. A difference in mitotic index (i.e., number of cells in mitosis divided by total cells) would indicate that the drug had an effect on proliferation. One may also be interested in the effect of a drug on proliferating cells, so that the outcome of the test will depend to the degree of proliferation that occurred during the test. There are many other situations where a simple method for measuring mitotic index without a significant effect on the purpose of the measurement would be of value.

Brief Description of Certain Relevant Literature

The detection of galactosidase and the use of galactosidase as a label is described in a large number of patents which describe chromogenic substrates, e.g., U.S. Pat. No. 4,978,613 to Bieniarz, et al. issued Dec. 18, 1990, entitled “Beta-lactamase assay employing chromogenic precipitating substrates;” U.S. Pat. No. 5,338,843 to Quante, et al., issued Aug. 16, 1994, entitled “Fluorogenic and chromogenic β-lactamase substrates,” as well as U.S. Pat. No. 5,583,217, “Fluorogenic and β lactamase substrates;” U.S. Pat. No. 5,741,657, “Fluorogenic substrates for β-lactamase and methods of use;” U.S. Pat. No. 5,955,604, “Substrates for β lactamase and uses thereof;” U.S. Pat. No. 6,031,094, “Beta-lactam substrates and uses thereof;” U.S. Pat. No. 6,291,162, “Cytosolic forms of β-lactamase and uses thereof;” U.S. Pat. No. 6,472,205 “Cytosolic forms for β lactamase and uses thereof;” U.S. Patent application No. 2003/0003526, “Beta-lactamase substrates having phenolic ethers;” European Publication No. 0817785, “Substrates for Beta-lactamase and uses thereof;” European Publication No. 0553741, “Fluorogenic and chromogenic betalactamase substrates;” and European Publication No. 1081495, “Quenchers for fluorescence assays.”

The use, generally, of enzyme fragment complementation (“EFC”) in other, unrelated assays is described, for example, in US PGPUB 2003/0092070 by Zhao, et al., published May. 15, 2003, entitled “Genetic construct intracellular monitoring system;” US PGPUB 2004/0106158 by Naqvi, et al., published Jun. 3, 2004, entitled “IP3 protein binding assay;” US PGPUB 2004/0137480 by Eglen, published Jul. 15, 2004, entitled “Monitoring intracellular proteins;” US PGPUB 2005/0136488 by Horecka, et al., published Jun. 23, 2005, entitled “Cellular membrane protein assay;” US PGPUB 2006/0019285 to Horecka et al., published Jan. 26, 2006 entitled “Analysis of intracellular modifications,” U.S. Pat. No. 5,434,052 to Khanna, issued Jul. 18, 1995, entitled “Complementation assay for drug screening;” U.S. Pat. No. 5,037,735 to Khanna, et al., issued Aug. 6, 1991, entitled “Visual discrimination qualitative enzyme complementation assay;” and U.S. Pat. No. 5,244,785 to Loor, et al., issued Sep. 14, 1993, entitled “Determination of high molecular weight analytes using a β-galactosidase complementation assay.”

### SUMMARY OF THE INVENTION

The following brief summary is not intended to include all features and aspects of the present invention, nor does it imply that the invention must include all features and aspects discussed in this summary.

The present invention comprises methods employing enzyme fragment complementation (“EFC”) for measuring mitotic index of a cell culture. In EFC, the members of the pair are referred to as an enzyme donor (“ED”), which is arbitrarily the smaller member, and an enzyme acceptor (“EA”). Cells here will comprise one member of the pair of the EFC in the nucleus and the other member of the EFC pair in the cytosol. Upon undergoing mitosis, the two members (EA and ED) of the EFC pair come into complex formation. In the presence of a substrate that provides a detectable product the mitotic event can be determined.

### BRIEF DESCRIPTION OF THE DRAWINGS

FIG. 1 is a schematic diagram of the known mammalian cell cycle, showing compounds which act at two different stages to arrest/block mitosis;

FIG. 2 is set of photographs showing, by immunofluorescence, localization of EA (FIG. 2A) and GR-PL (FIG. 2B), where GR is a human glucocorticoid receptor fragment and PL is a β-galactosidase enzyme donor fragment, and wherein the cytoplasm can be seen to be stained green and the nuclei stained blue;

FIG. 3 is a bar graph showing the results of testing Clone #69 in response to cell cycle blocking compounds; and

FIG. 4 is a pair of photographs showing immunofluorescence of cell line CHO-K1+cyto-EA, with cytoplasm stained green and nuclei stained blue.

The patent or application file contains at least one drawing executed in color. Copies of this patent or patent application publication with color drawings will be provided to the office upon request and payment of the necessary fee.

### DESCRIPTION OF THE SPECIFIC EMBODIMENTS

Simple protocols for the determination of mitosis are provided employing enzyme fragment complementation (“EFC”). Cells are engineered to contain an ED and EA pair for EFC. The cells comprise one member of the EFC pair in the nucleus and the other member of the EFC pair in the cytosol. The members of the pair are referred to as enzyme donor (“ED”), which, where the two members are substantially different in size, is arbitrarily the smaller member, and enzyme acceptor (“EA”). The ED will generally be in the range of about 36 to 90, more usually about 40 to 60, amino acids. One of the members of the EFC pair is joined to a polypeptide sequence that causes the member to reside in the nucleus. The member is preferably the EA. The polypeptide sequence is termed the “NLS/NRS,” meaning either an NLS (nuclear localization signal), an NRS (nuclear retention signal), or both an NLS and NRS. The NLS/NRS member will be directed to the nucleus after translation in the cytoplasm.

A number of NLS and NRS sequences are known.

A nuclear localization signal (NLS) is a short stretch of amino acids that mediates the transport of nuclear proteins into the nucleus. Such sequences have been combined in tandem. Further examples of NLS sequences are given in “Finding nuclear localization signals,” Murat Cokol, Raj Nair & Burkhard Rost http://cubic.bioc.columbia.edu/papers/2000 nls/paper.html. One known NLS sequence is from SV40. The simian virus 40 large T antigen (SV40 T Ag) NLS seven amino acid sequence is the prototype of a classical monopartite NLS, as disclosed, for example, in Ilmarinen et al. “The monopartite nuclear localization signal of autoimmune regulator mediates its nuclear import and interaction with multiple importin α molecules,” FEBS Journal 273 (2006) 315-32. As is also disclosed in this publication, some NLS sequences are bipartite, and may be brought together, as is discussed below. Further examples of NLS sequences are given in Cokol et al., “Finding nuclear localization signals,” Proc. Nat. Acad. Sci. Vol. 96, Issue 1, 91-96, Jan. 5, 1999.

An “NRS” is a sequence which promotes protein-protein interactions and directs subcellular localization and—in certain situations—nucleocytoplasmic shuttling of individual proteins, such as the phosphoprotein SR, which contains an RS domain. The RS domain is extensively phosphorylated and directs the subcellular localization. Further details are given in Cazalla et al. “Nuclear Export and Retention Signals in the RS Domain of SR Proteins,” Mol Cell Biol. October 2002; 22(19): 6871-6882.

A commonly used NRS is the NRS sequence from SC35 (GenBank 600813, 600812), although other sequences are available. Suitable sequences are given, for example, in Cazalla et al., supra, which demonstrates the presence of a dominant nuclear retention signal in the RS domain of SC35.

In some cases proteins that do not have a consensus NLS may be used for directing the ED or EA member of the EFC pair to the nucleus. The other member will remain in the cytosol. Upon mitosis, with the breakdown of the nuclear membrane, the two members of the EFC pair are brought together. In the presence of a substrate providing a detectable product, the cells may be analyzed by detecting the product. Alternatively, the cells may be lysed without lysis of the nucleus and the amount of the EFC complex determined by use of a substrate providing a detectable product.

The cell(s) that are employed will be subject to genetic modification for expressing an EFC member that is directed and remains in the nucleus and the other EFC member that remains in the cytosol. These cells may be subject to prior treatment by being maintained in an appropriate medium, washing, exposure to one or more agents that affect the proteomic status of the cell, that is, activate and/or inhibit one or more pathways, and the like. When the cells are ready to be assayed, the cells are provided in an appropriate vessel, a controlled environment provided for the cells and the cells grown for a sufficient period to provide a readout of the level of mitosis. The cells are then lysed/permeablized in an appropriate medium with enzyme substrate where the dilution of the cell lysate substantially inhibits additional complex formation of the EFC members that does not already exist as a result of mitosis.

The cells employed are characterized by having two genetic expression constructs, one construct comprising a fusion protein of an EFC pair member fused to an NLS/NRS and the other construct expressing the other EFC pair member. The expression constructs will have transcriptional and translational regulatory regions, which may be inducible or constitutive.

Usually, the expression constructs will be associated with other functional genetic sequences, such as sequences for integration, sequences for maintenance as an extrachromosomal element, sequences for penetration of the cellular membrane (i.e. the layer which separates a cell's interior from its surroundings and controls what moves in and out), sequences for selection of cells comprising the expression construct(s), etc. One may have a cell with only one of the constructs and add the other construct for transient expression, have both constructs integrated into the genome or present as stable or unstable extrachromosomal elements, or have both constructs present as transient constructs. Each of these possibilities may be exploited in accordance with the purpose of the determination.

Also fused to one or both of the members of the EFC pair may be an epitope tag, so that the location of the member of the EFC pair may be determined independently. Epitope tags are readily available and a sequence of from about 10-30 amino acids will suffice, where the sequence is not normally found in the host cell and there is a convenient binding member, e.g., antibody for binding to the epitope tag and identifying its location. For detection, the antibody may be labeled, two antibodies may be used in sandwich assays, one to the tag and the other to the fusion protein, or other convenient assay protocol can be employed.

Usually, the cells will have at least about 80% of the total amount of each of the members of the EFC pair in a single compartment, preferably there being at least one, more preferably both, with at least about 90% of the total amount of the members of the EFC pair in a single compartment. The single compartment where a member resides may be the nucleus, or the cytoplasm.

A number of proteins associated with mitosis or phase cycle blocking are of interest. These proteins include cyclins (e.g., Cyclin A, Cyclin B, Cyclin D, Cyclin E, Cyclin F, transcription factors (e.g., p53, Rbl, c-Abl, EF-1), kinases (e.g., p34cdc2, wee-1, DNA-PK), phosphatases (e.g., cdc25B, cdc25C) and other accessory proteins (e.g., ATM, MDM2, HDAC). These proteins are normally localized to the nucleus, although certain proteins (e.g., MDM2 or ATM) may also be located in the cytoplasm under certain conditions. See, for example, Kao et al. “p34(Cdc2) kinase activity is excluded from the nucleus during the radiation-induced G(2) arrest in HeLa cells,” J Biol Chem. Dec. 3, 1999;274(49):34779-84.

By targeting these proteins, where these proteins are fusion proteins and will maintain one of the EFC pairs in a particular compartment, while the other member of the pair is in the other compartment, one can investigate the effect of such compound on the protein target and its effect on mitosis. Using another cell where the cell is negative in the target protein allows one to isolate the effect.

In carrying out the determination, the cells in an appropriate culture medium may be dispersed, adhering to the surface of a vessel or a combination thereof. A particular number of cells will be chosen which may be a single cell, at least ten cells, usually at least 10² cells and usually not more than about 10⁵, more usually not more than about 5×10⁴. The number of cells is not critical to this invention and will be selected in accordance with the purpose of the determination, the level of signal required, and other pragmatic considerations. The cells may be primary cells or cell lines, where the primary cells or cell lines may be genetically modified, as appropriate.

The cells may be grown in an appropriate growth medium for a reasonable period to stabilize the cells, provide for proliferation of the cells, the cells may be blocked in a particular phase, e.g., S-phase, provide for the cells to be in a particular metabolic or other status, cell cycle arrested, agonist or antagonist treated, serum starved, serum stimulated, etc. The environment may then be changed in accordance with the purpose of the assay. For example, if one is interested in the effect of a compound on mitosis, the compound would be added to the medium. Temperatures, concentrations, components of the medium, etc., may be changed in accordance with the purpose of the assay. Where inducible transcriptional regulatory regions have been used, the inducible gene(s) may be turned on or off, e.g., tet regulatory region.

After the cells have been subjected to the desired environment for a sufficient time period, e.g., incubated, the cells may then be assayed for their mitotic index.

If the assay is performed intracellularly, the signal from the cells can be determined in a variety of ways, e.g., calorimetrically, fluorometrically, such as fluorescence activated cell sorter, chemiluminescently, etc. A substrate is introduced into the cells, where the substrate is capable of transport across the cell membrane, the membrane is made permeable, e.g., by isotonic shock, or the like. Desirably, with a fluorescent product from the substrate, the product should have lower permeability than the substrate. Where the determination is made extracellularly, the cells are lysed in an appropriate lysing medium and the signal determined appropriately. The lysing involves substantial dilution of the cellular material, usually at least about 5-fold and may be 10-fold or more, usually not more than about 100-fold. The rapid dilution has the effect of substantially inhibiting forming new enzyme complexes not previously formed intracellularly. A single determination may be made or a plurality of determinations at different time periods from an initial event, e.g., termination of exposure to an environment, lysing, etc.

There are a number of ways in which the assay may be used. The assay may be used to determine whether changes in the environment, e.g., candidate agents or drugs, are able to affect mitosis. By using the subject assay with modified cells where one or more genes may be turned on or off, the effect of compounds on cells having the presence or absence of specific proteins can be established. One may also use RNAi, in conjunction with the subject assays to determine whether specific transcriptional and translational products affect mitosis. In the same way, one can establish pathways involved in mitosis and the pathway response to changes in the environment. All of these investigations follow normal testing procedures, e.g., high throughput screening, using the subject protocols and components in analogous ways. Usually, one will employ a control lacking the candidate agent and compare the result in the presence and absence of the candidate agent. A difference indicates that the candidate agent modulates mitosis. One may employ high throughput techniques such as fluorescence activated cell sorting, since a mitotic signal is either present or not in a cell, and there is no need to localize the signal to a particular cellular location.

The subject invention will generally have a fusion protein to maintain the ED in either the nuclear or, preferably, in the cytosol compartment and impart stability. The particular partner will be primarily arbitrarily chosen as one that does not interfere in the assay, maintains the fusion product in the selected compartment and is sufficiently stable to retain a sufficient concentration in the cell as to provide a robust signal. The shorter member of the EFC will usually be fused to an innocuous protein to enhance its stability. In view of the low molecular weight of the shorter member, it appears to be easily degraded, so as to substantially diminish its availability. Generally the protein will have a molecular weight of at least about 5 kD, usually at least about 10 kD, and generally less than about 50 kD. Proteins that have been used are extensively described in the literature and include such proteins as glutathione synthase, green fluorescent protein (GFP), maltose binding protein (MBP), annexin proteins, etc.

The first component of the subject invention is the fusion protein described above and its expression construct. The ED may be at either the C-terminus, the N-terminus or internal to the fusion protein. The particular site of the ED in the fusion protein will depend upon convenience, stability and retaining the ability of the fusion protein to complex with EA to form an active enzyme.

The ED may be inserted into the coding region in a variety of ways. For a cDNA gene construct, one may select a suitable restriction site for insertion of the sequence, where by using overhangs at the restriction site, the orientation is provided in the correct direction. Alternatively, one may use constructs that have homologous sequences with the target gene and allow for homologous recombination, where the homologous sequences that are adjacent in the target gene are separated by the ED in the construct. By using a plasmid in yeast having the cDNA gene, with or without an appropriate transcriptional and translational regulatory region, one may readily insert the ED construct into the cDNA gene at an appropriate site. Alternatively, one may insert the ED coding region with the appropriate splice sites in an intron or in an exon of the gene encoding the protein. In this way, one can select for a site of introduction at any position in the protein. In some instances, it will be useful to make a number of constructs, where the ED is introduced into an intron and test the resulting proteins for ED activity and retention of function of the protein. Various other conventional ways for inserting encoding sequences into a gene can be employed. The preferred ED and EA are derived from β glactosidase. The ED may be prepared from the N-terminal region of E. coli β galactosidase, Genbank Accession No. AAN78938, beginning, e.g., at residue 7, with the addition of an N terminal cysteine and a cysteine replacement for arginine near the C terminus. Other regions of the known β galactosidase sequence may be adapted for use as the ED.

For expression constructs and descriptions of other conventional manipulative processes, see, e.g., Sambrook, Fritsch & Maniatis, “Molecular Cloning: A Laboratory Manual,” Second Edition (1989) Cold Spring Harbor Laboratory Press, Cold Spring Harbor, N.Y. (herein “Sambrook et al., 1989”); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The gene encoding the fusion protein will be part of an expression construct. The gene is positioned to be under transcriptional and translational regulatory regions functional in the cellular host. The regulatory region may include an enhancer, which may provide such advantages as limiting the type of cell in which the fusion protein is expressed, requiring specific conditions for expression, naturally being expressed with the protein, and the like. In many instances, the regulatory regions may be the native regulatory regions of the gene encoding the protein, where the fusion protein may replace the native gene, may be in addition to the native protein, either integrated in the host cell genome or non-integrated, e.g., on an extrachromosomal element. The protein may be selected in relation to the desirability of its regulatory region or an exogenous regulatory region may be used.

It should be understood that the site of integration of the expression construct will affect the efficiency of transcription and, therefore, expression of the fusion protein. One may optimize the efficiency of expression by selecting for cells having a high rate of transcription, one can modify the expression construct by having the expression construct joined to a gene that can be amplified and co-amplifies the expression construct, e.g., DHFR in the presence of methotrexate, or one may use homologous recombination to ensure that the site of integration provides for efficient transcription. By inserting an insertion element, such as Cre-Lox at a site of efficient transcription, one can direct the expression construct to the same site. In any event, one will usually compare the β-galactosidase activity from cells in a predetermined environment to cells in the environment being evaluated. By appropriate choice of transcriptional regulatory region and site of integration, one can control the level of the fusion protein in the compartment where it is retained. Similarly, for the other member of the EFC pair, one can exploit the same considerations so as to have the desired level of the two members in the different compartments. For the most part, the fusion protein will comprise the ED or α-fragment of β-galactosidase.

There are a large number of commercially available transcriptional regulatory regions that may be used and the particular selection will generally not be crucial to the success of the subject invention. Also, the manner in which the fusion gene construct is introduced into the host cell will vary with the purpose for which the fusion gene is being used. The transcriptional regulatory region may be constitutive or inducible. In the former case, one can have a steady state concentration of the fusion protein and/or the other member of the EFC in the cells, while in the latter case one can provide going from the substantially total absence (there is the possibility of leakage) to an increasing amount of the fusion protein or other member of the EFC until a steady state is reached. With inducible transcription, one can cycle the cell from a state where the fusion protein is absent to a state where the steady state concentration of the fusion protein is present.

Copending application PGPUB 2003/0092070 entitled, “Genetic Construct Intracellular Monitoring System” (referenced in the Background hereof), has a large section on vectors for introduction of the constructs, methods for introducing the vectors, monitoring the transfection, transcriptional regulatory regions, namely promoters, strains of host cells that can find use, and other useful information related to the introduction of the constructs into cells, all of which is specifically incorporated herein by reference as if set forth fully here.

Briefly, the above-mentioned application refers in part to known vector systems such as a defective herpes virus 1 (HSV1) vector (Kaplitt et al., 1991, Molec. Cell. Neurosci. 2:320-330); an attenuated adenovirus vector, such as the vector described by Stratford-Perricaudet et al. (1992, J. Clin. Invest. 90:626-630 a defective adeno-associated virus vector (Samulski et al., 1987, J. Virol. 61:3096-3101; Samulski et al., 1989, J. Virol. 63:3822-3828). Alternatively, “naked DNA” constructs may be used; alternatively a DNA vector transporter may be used (see, e.g., Wu et al., 1992, J. Biol. Chem., 267:963-967; Wu and Wu, 1988, J. Biol. Chem. 263:14621-14624; Hartmut et al., Canadian Patent Application No. 2,012,311, filed March 15, 1990). A number of commercial mammalian vectors are available with different capabilities, different promoters, msc's, and selection genes. pYACneo (Replicon), pAdvantage, pSI(SV40p), pTarget, pGIneo (Promega), Vitality hrGFP (Stratagene), pCMS-EGFP-1, pEGFP-NI (BD Biosciences), pVITROms (Invivogen), pRK-5 GFP (Fujisawa) and pCruz 22 (Santa Cruz) (supplier).

For convenience, various components of the subject assays may be provided in kits. For example, DNA constructs may be provided on the same or different vectors to express the components of the EFC assay. Alternatively, cells containing the constructs may be provided, where the cells are either genetically modified or unmodified from the natural cells or cells strains, e.g., inhibiting or activating a particular gene(s) or introduction of a gene(s) that is not expressed by the cell. In addition, buffers may be included, culture media, assay substrate to measure EFC activity can be provided, etc.

The following examples are offered by way of illustration and not by way of limitation.

### Experimental

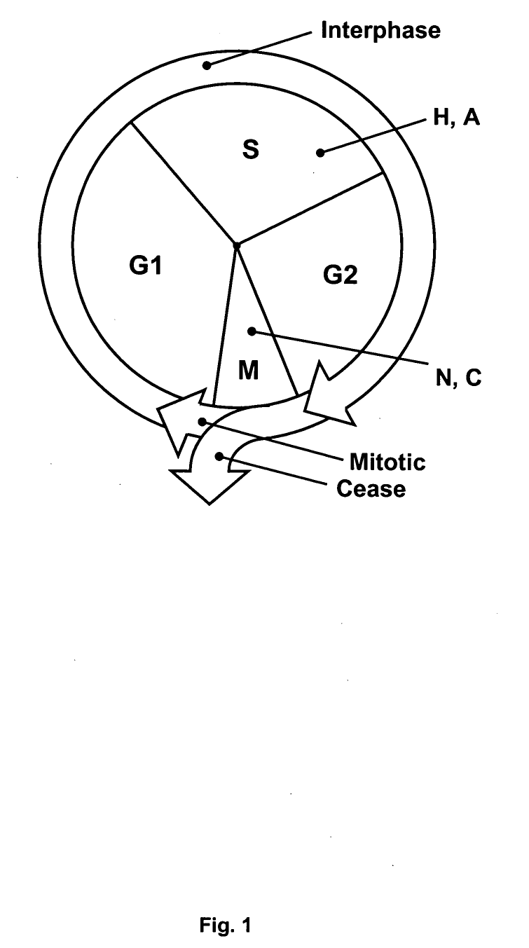

A series of different compounds that block at different stages of the cell cycle were tested. Vinblastine, colchicine, nocodazole and paclitaxel (Taxol®) all arrest the cell in the G2/M phases by acting on microtubule formation and organization. Hydroxyurea and aphidicolin block the cell cycle in S-phase by effecting DNA replication (FIG. 1).

FIG. 1 represents a known diagram of a eukaryotic cell cycle showing mitosis. Mitosis is nuclear division plus cytokinesis, and produces two identical daughter cells during prophase, prometaphase, metaphase, anaphase, and telophase. Interphase, shown above the mitotic region, is often included in discussions of mitosis, but interphase is technically not part of mitosis, but rather encompasses stages G1, S, and G2 of the cell cycle. FIG. 1 shows drugs H and A (hydroxyurea and aphidicolin) acting in S phase, and drugs N, and C (nocodazol and colchicine) acting in the “M” phase, which is mitosis. Other drugs, such as taxol and viblastine are known to act in different phases of the cell cycle, depending on the cell type. For example, taxol acts in M phase in T47D breast cancer cells. As is shown, cells may either continue to divide (“Mitotic”) or cease division (“Cease”).

In all experiments described, 20,000 cells/well were plated in a 96 well Corning clear bottom white plate in a total volume of 100 μL. The cells were treated for 24 hours with 5 μL of either the appropriate vehicle control or varying concentrations of the six compounds listed above. The next day, 100 μL of Tropix/ABI Gal screen cell lysis buffer/substrate mixture (24:1 ratio of component) was added to the cells/media and the plate was read on the Victor II luminescent plate reader at 30, 60 and 120 minutes after the lysis/substrate addition.

### EXAMPLE

### Example 1

The initial test of the cell cycle arresting compounds was performed on a double stable cell line having both of the constructs expressing the EA-NLS/NRS and GR-PL. (PL is β-galactosidase enzyme donor fragment and EA is the enzyme acceptor fragment available from DiscoveRx, Fremont, CA.). The parental line, C2C12 is derived from mouse muscle cells. In the experiments in which EA-NLS/NRS and GR-PL are expressed in the C2C12 parental cell line, the constructs were generated by subcloning the human GR sequence into a MFG-based retroviral vector that had been molecularly altered in a lab at Stanford. An MFG vector is described in U.S. Pat. No. 6,544,771. The EA-NLS/NRS fragment was subcloned into a wzl-based retroviral vector again molecularly altered in a lab at Stanford. In the experiments performed using a CHO-K1 parental cell line background the EA-NLS/NRS was subcloned into the Kpn I and Xba I sites of pcDNA3.1 Hygro vector from Invitrogen (catalog #V870-20). The plasmid was introduced into the cells via FuGene6 (Roche) transfection reagent. Cells were selected in the presence of 250 μg/mL of Hygromycin and single cell clones isolated that expressed the EA-NLS/NRS. The human GR gene was cloned by PCR and subcloned into the Xho I and Bam HI sites of the DiscoveRx vector-pCMV-myc-PL (C3). The plasmid was introduced into the selected EA-NLS/NRS expressing clone isolated above by FuGene 6 transfection. Another round of screening in the presence of 300-500 μg/mL of G418 was used to select GR-PL transfected clones. Clonal selection was performed to finally identify the clone that was used in these studies. In the studies using the CHO-K1+cyto-EA and cJUN-PL, the same Invitrogen pcDNA3.1 Hygro vector was used to express EA. In this case, the EA fragment was subcloned into the Kpn I/Not I sites of pcDNA3.1 Hygro. The plasmid DNA was introduced as described above using FuGene6 reagent. Cells were selected in the presence of 250 μg/mL of hygromycin and clonal selection was performed. The c-Jun gene was generated by PCR using an existing template copy of the gene and then subcloned into the Xho I/Bam HI sites of pCMV-PL-myc (C3).

In these cells, EA is localized in the nucleus (EA-NLS/NRS), while PL (a 55 mer α-fragment of β-galactosidase, SEQ ID NO: 1; fused to the human glucocorticoid receptor was retained in the cytoplasm (GR-PL)(>pCMV-PL\C3\Myc\(nuc) SEQ ID NO: 2. An inert fragment of the glucocorticoid receptor (GR) was chosen from a number of possible cytoplasmic proteins, including the hormone receptors, for use in fusing to the ED to prevent protease degradation or other instability of the ED. The cells were treated and assayed as described above. As seen in TABLE 1, Nocodazole treatment (1-10 μg/mL) showed a ˜2-fold increase in EFC activity, whereas, e.g. vinblastine, which does not act in M phase, showed no increase in EFC activity.

30 min read/Stanford GR cells

| | | | | | | |
|---|---|---|---|---|---|---|
| | | | | | | % |
| R1 | R2 | R3 | Avg | Ratio | SD | CV |

Taxol

| | | | | | | | |
|---|---|---|---|---|---|---|---|
| Conc (μM) | | | | | | | |
| 0 | 1774 | 2795 | 3056 | 2542 | 1 | 678 | 27 |
| 0.03 | 2624 | 3115 | 3543 | 3094 | 1 | 460 | 15 |
| 0.1 | 2602 | 4110 | 4340 | 3684 | 1 | 944 | 26 |
| 0.3 | 2638 | 3361 | 3639 | 3213 | 1 | 517 | 16 |
| 1 | 3016 | 3955 | 4104 | 3692 | 1 | 590 | 16 |

Avg % CV = 20

Nocodazole

| | | | | | | | |
|---|---|---|---|---|---|---|---|
| Conc (μg/mL) | | | | | | | |
| 0.0 | 2849 | 3739 | 3274 | 3287 | 1 | 445 | 14 |
| 0.3 | 4243 | 4486 | 4599 | 4443 | 1 | 182 | 4 |
| 1.0 | 6029 | 6355 | 6678 | 6354 | 2 | 325 | 5 |
| 3.3 | 5669 | 6175 | 6360 | 6068 | 2 | 358 | 6 |
| 10.0 | 4401 | 7395 | 5806 | 5867 | 2 | 1498 | 26 |

Avg % CV = 11

Aphidicolin

| | | | | | | | |
|---|---|---|---|---|---|---|---|
| Conc (μM) | | | | | | | |
| 0.0 | 2782 | 2985 | 3178 | 2982 | 1 | 198 | 7 |
| 0.3 | 3081 | 3752 | 3500 | 3444 | 1 | 339 | 10 |
| 1.0 | 3250 | 3284 | 3388 | 3307 | 1 | 72 | 2 |
| 3.3 | 3171 | 3121 | 3105 | 3132 | 1 | 34 | 1 |
| 10.0 | 2827 | 3648 | 3765 | 3413 | 1 | 511 | 15 |

Avg % CV = 7

Vinblastine

| | | | | | | | |
|---|---|---|---|---|---|---|---|
| Conc (μg/mL) | | | | | | | |
| 0.0 | 10819.0 | 11743 | 9202 | 10588 | 1 | 1286 | 12 |
| 1.0 | 10417.0 | 10773 | 11042 | 10744 | 1 | 314 | 3 |
| 3.3 | 10894.0 | 11873 | 13468 | 12078 | 1 | 1299 | 11 |
| 10.0 | 10269.0 | 10703 | 11281 | 10751 | 1 | 508 | 5 |
| 30.0 | 10692.0 | 10910 | 11196 | 10933 | 1 | 253 | 2 |

Avg % CV = 7

Colchicine

| | | | | | | | |
|---|---|---|---|---|---|---|---|
| Conc (μM) | | | | | | | |
| 0.0 | 11761 | 9069 | 9123 | 9984 | 1 | 1539 | 15 |
| 0.03 | 10442 | 12449 | 12456 | 11782 | 1 | 1161 | 10 |
| 0.1 | 6763 | 10024 | 10724 | 9170 | 1 | 2114 | 23 |
| 0.3 | 11132 | 10438 | 12160 | 11243 | 1 | 866 | 8 |
| 1.0 | 11891 | 12491 | 11866 | 12083 | 1 | 354 | 3 |

Avg % CV = 12

Hydroxyurea

| | | | | | | | |
|---|---|---|---|---|---|---|---|
| Conc (μg/mL) | | | | | | | |
| 0.0 | 11553 | 10363 | 10532 | 10816 | 1 | 644 | 6 |
| 1.0 | 12080 | 11562 | 11424 | 11689 | 1 | 346 | 3 |
| 3.3 | 12588 | 10806 | 11171 | 11522 | 1 | 941 | 8 |
| 10.0 | 8813 | 9401 | 9419 | 9211 | 1 | 345 | 4 |
| 30.0 | 7272 | 7226 | 7165 | 7221 | 1 | 54 | 1 |

Avg % CV = 4

TABLE 1 above shows the results of a series of experiments determining the average readout of luminescence with different drugs with a given coefficient of variance (% CV) from testing cell cycle blocking compounds on C2C12+EA-NLS/NRS+GR-PL cells, i.e., the mouse muscle cell line C2C12 engineered with an enzyme acceptor/nuclear location signal and the glucocoticoid receptor and enzyme donor fragment PL.

### Example 2

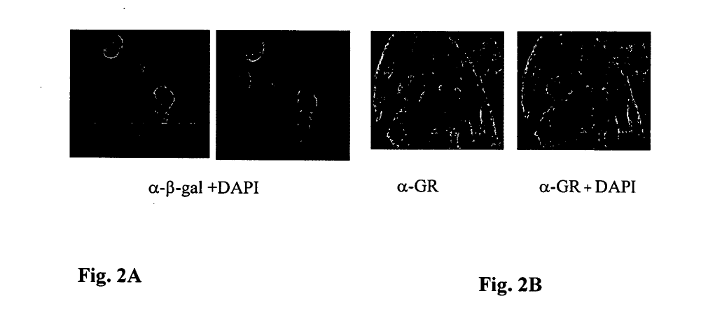

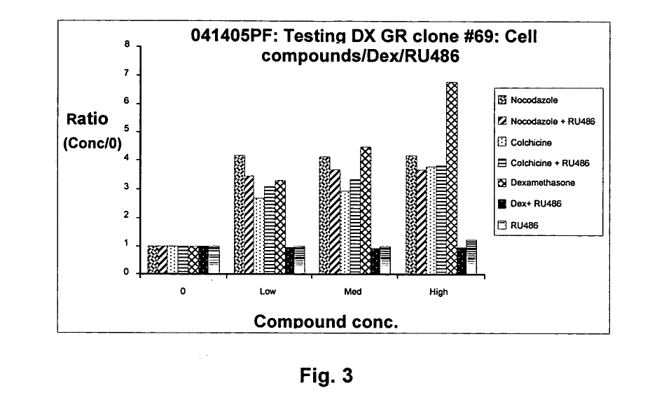

In the next experiment, an antibiotic selected pool population of CHO-K1 cells that express EA-NLS/NRS and GR-PL were tested with the same six set of cell cycle blocking compounds. These cells have been characterized by immunofluorescence using antibodies specific to EA-NLS/NRS (Promega monoclonal antibody to beta galactosidase) and GR (Abcam polyclonal antibody) and show that greater than 90% of EA-NLS/NRS is found localized in the nucleus (see FIG. 3a) and greater than 80% of the GR is found in the cytoplasm (see FIG. 3b). FIGS. 2a and 2b show the immunofluorescence localization of EA and GR-PL in that the blue DAPI nuclear staining can be seen to be concentrated in the nucleus, while the green fluorescein stain on the antibody (from Abcam PLC) to the glucocorticoid receptor is seen in the cytoplasm. TABLE 2 shows the data from the testing of the CHO-K1+EA-NLS/NRS+GR-PL cells. Again, six tables are presented one for each of the six drugs tested.

30 min read/DX M19/GR (pool)

| | | | | | | |
|---|---|---|---|---|---|---|
| | | | | | | % |
| R1 | R2 | R3 | Avg | Ratio | SD | CV |

Taxol

| | | | | | | | |
|---|---|---|---|---|---|---|---|
| Conc (μM) | | | | | | | |
| 0 | 5441 | 6673 | 7700 | 6605 | 1 | 1131 | 17 |
| 0.03 | 7314 | 7582 | 6605 | 7167 | 1 | 505 | 7 |
| 0.1 | 10680 | 9945 | 9258 | 9961 | 2 | 711 | 7 |
| 0.3 | 11709 | 8906 | 7904 | 9506 | 1 | 1972 | 21 |
| 1 | 17111 | 16460 | 14344 | 15972 | 2 | 1447 | 9 |

Avg % CV = 12

Nocodazole

| | | | | | | | |
|---|---|---|---|---|---|---|---|
| Conc (μg/mL) | | | | | | | |
| 0.0 | 9304 | 10557 | 8127 | 9329 | 1 | 1215 | 13 |
| 0.3 | 15003 | 17995 | 13585 | 15528 | 2 | 2251 | 14 |
| 1.0 | 23225 | 26942 | 24165 | 24777 | 3 | 1933 | 8 |
| 3.3 | 24010 | 24116 | 23172 | 23766 | 3 | 517 | 2 |
| 10.0 | 22978 | 25565 | 28584 | 25709 | 3 | 2806 | 11 |

Avg % CV = 10

Aphidicolin

| | | | | | | | |
|---|---|---|---|---|---|---|---|
| Conc (μM) | | | | | | | |
| 0.0 | 8114 | 9939 | 10512 | 9522 | 1 | 1252 | 13 |
| 0.3 | 5478 | 5578 | 5469 | 5508 | 1 | 61 | 1 |
| 1.0 | 5656 | 5666 | 5491 | 5604 | 1 | 98 | 2 |
| 3.3 | 4392 | 4542 | 4749 | 4561 | 0 | 179 | 4 |
| 10.0 | 7213 | 6958 | 6651 | 6941 | 1 | 281 | 4 |

Avg % CV = 5

Vinblastine

| | | | | | | | |
|---|---|---|---|---|---|---|---|
| Conc (μg/mL) | | | | | | | |
| 0.0 | 5586 | 5151 | 4385 | 5041 | 1 | 608 | 12 |
| 1.0 | 4205 | 4523 | 4277 | 4335 | 1 | 167 | 4 |
| 3.3 | 4410 | 4190 | 4436 | 4345 | 1 | 135 | 3 |
| 10.0 | 3907 | 4126 | 4201 | 4078 | 1 | 153 | 4 |
| 30.0 | 5161 | 6014 | 6583 | 5919 | 1 | 716 | 12 |

Avg % CV = 7

Colchicine

| | | | | | | | |
|---|---|---|---|---|---|---|---|
| Conc (μM) | | | | | | | |
| 0.0 | 3051 | 2690 | 2651 | 2797 | 1 | 221 | 8 |
| 0.03 | 3128 | 3381 | 3727 | 3412 | 1 | 301 | 9 |
| 0.1 | 6507 | 6048 | 4886 | 5814 | 2 | 836 | 14 |
| 0.3 | 7078 | 7335 | 7341 | 7251 | 3 | 150 | 2 |
| 1.0 | 10671 | 11680 | 13178 | 11843 | 4 | 1261 | 11 |

Avg % CV = 9

Hydroxyurea

| | | | | | | | |
|---|---|---|---|---|---|---|---|
| Conc (μg/mL) | | | | | | | |
| 0.0 | 3303 | 3851 | 4203 | 3786 | 1 | 454 | 12 |
| 1.0 | 4403 | 4542 | 4740 | 4562 | 1 | 169 | 4 |
| 3.3 | 4423 | 4439 | 4304 | 4389 | 1 | 74 | 2 |
| 10.0 | 3865 | 4116 | 3897 | 3959 | 1 | 137 | 3 |
| 30.0 | 3825 | 3770 | 3785 | 3793 | 1 | 28 | 1 |

Avg % CV = 4

As shown by the increased average fluorescence from the cleavage of the active, complemented βGal substrate overnight treatment with Taxol, nocodazole and colchicine resulted in as great as a 4-fold increase in EFC that was titrated with increasing concentrations of each of these compounds. As predicted, both aphidicolin and hydroxyurea did not cause an increase in EFC activity. These results suggest that the compounds that do not affect the events of nuclear envelope breakdown (i.e., the release of EA from the nucleus) but still cause an arrest in cell cycle progression did not result in the complementation of EA from the nucleus with the GR-PL that is localized in the cytoplasm to produce an active enzyme complex that can turn over the β-galactosidase chemiluminescent substrate. This only occurs with compounds that block the cells in mitosis, allowing EA and ProLabel to complement.

### Example 3

In the next experiment, a stable clone (clone #69) expressing both EA-NLS/NRS and GR-PL was isolated in a CHO-K1 parental background. To demonstrate the specificity of the cell cycle blocking compounds, pre-incubation in the presence of RU486 (a specific antagonist of GR) was tested. 20,000 cells/well were plated in a 96 well white coming multi-well plate and allowed to adhere overnight. The next day, the cells were washed two times with serum free F12 media and 100 μL of serum free F12 media was added to the cells. The cells were then incubated in either vehicle (ethanol-1% final concentration) or 10 μM RU486 for one hour. To the cells, three different concentrations of dexamethasone (300, 100, 30 μM) (an agonist of GR), RU486 (30, 10, 3.33 μM), colchicine (1, 0.3, 0.1 μ/mL) or nocodazole (10, 3.33, 1.11 μg/mL) were added and the incubation went overnight at 37° C. with 5% CO₂. The next day, the media was aspirated off and 100 μL of Tropix/ABI Gal screen cell lysis/substrate reagent was added to the cells. The plate was read on the Victor II reader at 30, 60 and 120 minutes.

Results are shown in FIG. 3 as ratios of fluorescence to drug concentration (0, low medium and high) as well as tables for seven drugs tested. As shown in FIG. 3, the cells showed a very strong response (increased EFC activity) to the dexamethasone titration that was blocked by the incubation with RU486. Although RU486 can act as a weak agonist on its own, it did not show an increase in EFC activity when titrated. Both nocodazole and colchicine showed an increase in EFC activity (˜3-4 fold) at each of the concentrations tested. This increase in EFC response was not blocked by the incubation with RU486, suggesting the response is not related to the nuclear translocation response of the GR. These results further support that the increase in EFC activity observed by the addition of the cell cycle arresting compounds was due to breakdown of the nuclear envelope and subsequent release of the EA to the cytoplasm where it can complement with the GR-PL present and turn over substrate. These results are further presented in TABLE 3 below:

Clone #69

| | | | | | |
|---|---|---|---|---|---|
| Conc | R1 | R2 | R3 | Avg | Ratio |

Nocodazole

| | | | | | |
|---|---|---|---|---|---|
| 0 | 3346 | 2737 | 2219 | 2767 | 1 |
| Low | 11488 | 11630 | 11606 | 11575 | 4 |
| Med | 11364 | 11514 | 11457 | 11445 | 4 |
| High | 11504 | 11657 | 11616 | 11592 | 4 |

Nocodazole/+RU486

| | | | | | |
|---|---|---|---|---|---|
| 0 | 3179 | 4433 | 4438 | 4017 | 1 |
| Low | 11466 | 16071 | 14099 | 13879 | 3 |
| Med | 15554 | 15744 | 12922 | 14740 | 4 |
| High | 16620 | 14877 | 13043 | 14847 | 4 |

Colchicine

| | | | | | |
|---|---|---|---|---|---|
| 0 | 466 | 444 | 371 | 427 | 1 |
| Low | 1274 | 1126 | 1007 | 1136 | 3 |
| Med | 1345 | 1304 | 1073 | 1241 | 3 |
| High | 1850 | 1695 | 1323 | 1623 | 4 |

Colchicine/+RU486

| | | | | | |
|---|---|---|---|---|---|
| 0 | 5578 | 5606 | 4110 | 5098 | 1 |
| Low | 16801 | 18288 | 11942 | 15677 | 3 |
| Med | 18756 | 19794 | 12588 | 17046 | 3 |
| High | 19868 | 22803 | 15717 | 19463 | 4 |

Dexamethasone

| | | | | | |
|---|---|---|---|---|---|
| 0 | 394 | 345 | 401 | 380 | 1 |
| Low | 1528 | 1230 | 1016 | 1258 | 3 |
| Med | 2033 | 1768 | 1290 | 1697 | 4 |
| High | 3173 | 2571 | 1934 | 2559 | 7 |

Dexamethasone/+RU486

| | | | | | |
|---|---|---|---|---|---|
| 0 | 3013 | 3216 | 2508 | 2912 | 1 |
| Low | 2891 | 3057 | 2367 | 2772 | 1 |
| Med | 3020 | 2663 | 2370 | 2684 | 1 |
| High | 2917 | 2792 | 2752 | 2820 | 1 |

RU486

| | | | | | |
|---|---|---|---|---|---|
| 0 | 2430 | 2354 | 2376 | 2387 | 1 |
| Low | 2692 | 2259 | 2298 | 2416 | 1 |
| Med | 2578 | 2379 | 2249 | 2402 | 1 |
| High | 3199 | 2790 | 2657 | 2882 | 1 |

### Example 4

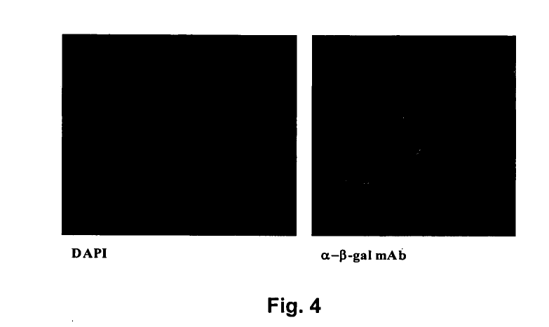

To further test the concept of sequestering of one β-galactosidase enzyme fragment in the nucleus (in this case PL) while localizing the other component in the cytoplasm (in this case EA) the following experiment was carried out. A CHO-K1 stable cell line that expressed EA (cyto-EA) that was localized in the cytoplasm (greater than 70% as seen in FIG. 4a) was transfected with cJUN-PL. It has been observed that cJUN-PL when transiently transfected into CHO-K1 cells almost exclusively localizes in the nucleus. The cyto-EA cells were transiently transfected with cJUN-PL plasmid DNA. Two days after the transfection, the cells were re-plated into a 96 well Corning white clear bottom multiwell plate at 20,000 cells/well. The cells were allowed to adhere overnight and the next day were treated with titrating concentrations of the six different cell cycle blocking compounds. The incubation was carried out overnight. The next day the media was removed from the cells and 100 μL of Tropix/ABI Gal screen cell lysis/substrate reagent was added to the cells. As seen in FIG. 4b, both nocodazole and colchicine addition caused a ˜2.1 fold increase in EFC activity. Both aphidicolin and hydroxyurea addition resulted in a negligible increase in EFC activity, suggesting background activity. These data are further presented in TABLE 4 below:

| | | | | | |
|---|---|---|---|---|---|
| Conc (μg/mL) | R1 | R2 | R3 | Avg | Ratio |

Nocodazole

| | | | | | |
|---|---|---|---|---|---|
| 0.0 | 7074 | 7998 | 6717 | 7263 | 1.0 |
| 3.0 | 9509 | 10402 | 9751 | 9887 | 1.4 |
| 10.0 | 11991 | 13821 | 12371 | 12728 | 1.8 |
| 30.0 | 10083 | 13241 | 12287 | 11870 | 1.6 |
| 100.0 | 13010 | 15624 | 16060 | 14898 | 2.1 |

Colchicine

| | | | | | |
|---|---|---|---|---|---|
| 0.0 | 6799 | 6653 | 7659 | 7037 | 1.0 |
| 0.3 | 7491 | 8424 | 9730 | 8548 | 1.2 |
| 1.0 | 11290 | 11068 | 9929 | 10762 | 1.5 |
| 3.0 | 12822 | 13938 | 13005 | 13255 | 1.9 |
| 10.0 | 13772 | 16082 | 14302 | 14719 | 2.1 |

Aphidicolin

| | | | | | |
|---|---|---|---|---|---|
| 0.0 | 9433 | 8581 | 8140 | 8718 | 1.0 |
| 1.5 | 7218 | 7840 | 7576 | 7545 | 0.9 |
| 4.4 | 9492 | 8842 | 9245 | 9193 | 1.1 |
| 13.3 | 6677 | 6620 | 7784 | 7027 | 0.8 |
| 40.0 | 8645 | 8931 | 9818 | 9131 | 1.0 |

Hydroxyurea

| | | | | | |
|---|---|---|---|---|---|
| 0.0 | 6318 | 6369 | 5412 | 6033 | 1.0 |
| 3.0 | 7319 | 8251 | 6690 | 7420 | 1.2 |
| 10.0 | 8237 | 7967 | 6699 | 7634 | 1.3 |
| 30.0 | 7217 | 7112 | 6624 | 6984 | 1.2 |
| 100.0 | 7955 | 7787 | 7483 | 7742 | 1.3 |

Conclusion

It is evident from the above results that the subject compositions and methods provide a rapid and convenient method to identify the effect of changes in environment, particularly candidate drugs, on mitosis. The method also allows the identification of proteins involved in the phase cycle and how they may affect the cycle going through mitosis. The method provides for a robust signal and there is little interfering background.

All publications and patent applications cited in this specification are herein incorporated by reference as if each individual publication or patent application were specifically and individually indicated to be incorporated by reference.

The above specific description is meant to exemplify and illustrate the invention and should not be seen as limiting the scope of the invention, which is defined by the literal and equivalent scope of the appended claims. Any patents or publications mentioned in this specification are indicative of levels of those skilled in the art to which the patent pertains and are intended to convey details of the invention which may not be explicitly set out but which would be understood by workers in the field. Such patents or publications are hereby incorporated by reference to the same extent as if each was specifically and individually incorporated by reference, as needed for the purpose of describing and enabling the method or material referred to.

What is claimed is:

1. A method for detecting mitosis employing members of enzyme fragmentation complex pairs capable of complexing to form an active enzyme, said members being an enzyme donor and an enzyme acceptor, wherein one of said members is in the cytosol and the other of said members is in the nucleus of a cell, said method comprising:
growing said cells to allow for mitosis to occur; and
measuring enzyme activity with a detectable substrate;
wherein a level of enzyme activity is a measure of the amount of mitosis.

2. A method according to claim 1, wherein said members are fragments of β-galactosidase.

3. A method according to claim 2 wherein one of the fragments is a substantially smaller fragment than the other and is fused to a protein normally found in the compartment in which said smaller fragment resides.

4. A method according to claim 1, wherein one of said members is fused to an NLS/NRS coding sequence.

5. A method according to claim 4, wherein said members independently complex

6. A method according to claim 1, wherein said measuring comprises lysing the cells, adding a substrate that forms a detectable product, and determining the detectable product.

7. A method for determining the effect of a candidate agent on mitosis employing members of enzyme fragmentation complex pairs capable of independently complexing to form an active β-galactosidase enzyme, said members being an enzyme donor and an enzyme acceptor, wherein said enzyme donor member is in the cytosol and said enzyme acceptor member is in the nucleus of a cell, said method comprising:
growing said cells to allow for mitosis to occur in the presence and absence of said candidate agent; and
measuring enzyme activity of the cells in the presence and absence of said candidate agent with a detectable substrate;
wherein a difference in level of enzyme activity in the presence and absence of said agent is a measure of the effect of said agent on mitosis.

8. A method for determining the effect of a candidate agent on mitosis in a cell having a cellular membrane, a nucleus and cytosol, employing members of enzyme fragmentation complex pairs capable of independently complexing to form an active β-galactosidase enzyme, said members being an enzyme donor and an enzyme acceptor, wherein said enzyme donor member is in the cytosol and said enzyme acceptor member is in the nucleus, said method comprising:
growing said cells to allow for mitosis to occur in the presence and absence of said candidate agent;
introducing a detectable substrate into said cell under conditions where said substrate is capable of transport across the cellular membrane; and
measuring the enzyme activity of the cells in the presence and absence of said candidate agent with said detectable substrate;
wherein the difference in level of enzyme activity in the presence and absence of said agent is a measure of the effect of said agent on mitosis.
